(19) United States
(12) Patent Application Publication
(10) Pub. No.: US 2026/0166100 A1
(43) Pub. Date: Jun. 18, 2026

(54) LACTIPLANTIBACILLUS PLANTARUM SUBSP. PLANTARUM STRAIN, AND BACTERIAL MEMBERANE VESICLE OF LACTIPLANTIBACILLUS PLANTARUM SUBSP. PLANTARUM STRAIN, AND USE OF THE SAME FOR IMPROVEMENT OF EYE DISEASE

(71) Applicants: TAIWAN PURITIC CORP., Hsinchu County (TW); CHINA MEDICAL UNIVERSITY, Taichung City (TW)

(72) Inventors: Lei Wan, Taichung City (TW); Hui-Ju Lin, Taichung City (TW); Yu-An Hsu, Taichung City (TW); Chi-Fong Lin, Miaoli County (TW)

(21) Appl. No.: 18/979,082

(22) Filed: Dec. 12, 2024

(51) Int. Cl. A61K 35/742; A61P 27/10; C12N 1/20; C12R 1/225

(52) U.S. Cl. CPC A61K 35/742; A61P 27/10; C12N 1/205; C12R 2001/225

(57) ABSTRACT

A Lactiplantibacillus plantarum subsp. plantarum strain, and a bacterial membrane vesicles derived from Lactiplantibacillus plantarum subsp. plantarum strain, and uses of the same for improvement of eye diseases are provided. The Lactiplantibacillus plantarum subsp. plantarum strain EP21 and the bacterial membrane vesicles are derived from Lactiplantibacillus plantarum subsp. plantarum strain EP21. Both the Lactiplantibacillus plantarum subsp. plantarum strain EP21 and the bacterial membrane vesicles derived from Lactiplantibacillus plantarum subsp. plantarum strain EP21 can inhibit inflammation by suppressing inflammatory factors and improve eye diseases effectively.

### FIELD OF THE INVENTION

The present invention relates to a Lactiplantibacillus plantarum subsp. plantarum strain and bacterial membrane vesicles (MV) derived from Lactiplantibacillus plantarum subsp. plantarum strain, especially to a composition containing Lactiplantibacillus plantarum subsp. plantarum strain EP21 and a composition containing bacterial MVs derived from the Lactiplantibacillus plantarum subsp. plantarum strain EP21 and uses of the same for improvement of eye diseases.

### STATEMENT REGARDING SEQUENCE LISTING

The sequence listing associated with this application is provided in text format in lieu of a paper copy and is hereby incorporated by reference into the specification. The name of the XML file containing the sequence listing is sl. The XML file is 5,138 bytes; was created on Jan. 22, 2025.

### BACKGROUND OF THE INVENTION

Myopia is growing around the world. There is an epidemic of myopia in East Asia. Recent publication estimates that by 2050, almost half the world population (49.8% (95% CI 43.4-55.7)) will have myopia (≤−0.5 D), and 9.8% (95% CI 5.7-19.4) high myopes (≤−5 D). High myopia is one of the important risk factors of blindness because it increases risk of retinal detachment, macular choroidal degeneration, premature cataracts, and glaucoma. The occurrence probability of neovascularization in patients with high myopia is 9 times higher than that in normal people. In Taiwan, the prevalence of myopia in 6-year-old children is 9.4% and reaches 75% in 15-year-old teenagers. The prevalence increases to 80%˜90% in 18-year-old adolescents and 10%˜20% of people in this age are highly myopic. Thereby prevention of myopia to avoid blindness has become an important global issue.

Axial myopia is the most common type observed in myopia. Owing to excessive elongation of vitreous chamber, there is an increase in axial length over an increase in corneal refractive power. Moreover, sclera tissue remodeling is associated with axial length elongation and myopia. This is due to reduced synthesis of the connective tissue and enhanced degradation of Type I collagen. During progression of myopia, composition and ductility of sclera have changed. However, precise pathogenesis of myopia remains unclear. For example, atropine, dopamine, pirenzepine, and 7-methylxanthine can relay myopia progression but underlying molecular mechanisms is still unknown. Both environmental and genetic factors are related to the development of myopia. In recent research, the environmental factor has important impact on the myopia progression. Many hypotheses predict the genetic factor affects molecular mechanism of myopia development but the environmental factor (such as outdoor time for activities) has no direct effects on myopia. Research available now indicate that retinal signaling plays a key role in adjustment of eyeball growth and axis. Signals that accelerate sclera tissue remodeling come from retina and affect photoreceptors and retinal pigment epithelium (RPE). Features of myopia include longer axial length (longer eyeballs), deeper vitreous chambers, thinner lenses, and flatter corneas.

Besides environmental and genetic factors, it is found that inflammation is associated with the development of myopia. The first paper mentioned about myopia and inflammation is published in 2011. The article reviewed the correlation between myopia and intraocular inflammation. Certain reports imply that myopia and allergic conjunctivitis are related. In techniques available now, atropine is widely used to slow myopia progression in clinic by reducing expression levels of c-Fos, NFκB, IL-6, and TNF-α which involve in chronic inflammation. The paper has been cited by American Optometric Association to show that atropine may reduce the progression of myopia by inhibiting of inflammation. In animal models of allergic conjunctivitis, allergic inflammation (especially allergic conjunctivitis) increases myopia development. Moreover, the progression of myopia is accelerated by direct application of PM2.5 to an eyeball surface of Syrian hamsters. Thus exposure to PM2.5 not only increases eye inflammation but also induce myopia. Some research now found that retina dopamine is recognized as a stop signal for normal growth of eyes and playing a role in development of myopia. Acupuncture can alleviate inflammation by activation of dopa decarboxylase which leads to higher dopamine level. Dopamine can inhibit expression of TNF-α and IL6 and further inhibit inflammation. That means acupuncture inhibits myopia development by suppressing inflammation which is initiated by activation of the dopamine-DIR signaling pathway. Now there are several anti-inflammatory medicines that inhibit myopia development such as resveratrol, dicoumarol, and herb formula containing two different herbs. Other research staff also reported that feeding mice with bovine lactoferrin prevents the onset of lens-induced myopia in mice through the IL-6-MMP-2 axis. Moreover, in clinical tests, crocetin which is a potent anti-inflammatory compound is used to treat children myopia and results show that crocetin has a suppressive effect on myopia progression in children. In 2021, inflammation is one of the IMI (International Myopia Institute) risk factors for myopia. Although the techniques available now provide clinical and experiment data showing a link between myopia and inflammation, sources of inflammatory reaction are still unknown. There is no disease or environmental factor which can induces such high incidence of myopia (in some area, the incidence rate of people under age 18 is over 90%).

It is reported that many different mediators are associated with development of myopia. In myopia, transforming growth factor beta (TGF-β) and matrix metalloproteinase-2 (MMP2) are increased while type I collagen is decreased. Transforming growth factor beta (TGF-β) signaling pathway involves a plurality of different reactions related to inflammatory reaction and tissue fibrosis and remodeling. Thereby TGF-β is closely associated with development of myopia. There are three different isomers of TGF-β in mammals. In eye tissue, TGF-β2 has the highest expression level. The expressions of TGF-β1, TGF-2, and TGF-3 are all positively correlated with optic axis.

Moreover, matrix metalloproteinases (MMPs) are responsible for degradation of ECM (extracellular matrix) proteins-collagen, gelatin, fibronectin, aggrecan, etc, and also working as a mediator of angiogenesis during inflammation. Thereby cooperation between the MMP family is considered to participate in pathogenic changes of myopia. In chicken sclera and treeshrew model, form deprivation myopia (FDM) results show that expression of MMP2 increases. In human samples, myopia eyes with higher expression of MMP2 have also been confirmed. Moreover, research indicates that TGF-β affects expression of MMP2 through activation of NFκB.

The complement system also participates in pathogenesis and mechanisms of myopia. In patients with pathologic myopia (−8 D˜−25 D), expression levels of C3 (p=0.004) and CH50 (p<0.001) are raised dramatically. In sclera of guinea pigs with negative lens defocused myopia, expression levels of C1q, C3, and C5b-9 are increased significantly. CD55 inhibits C3 and C5 convertase activity by inhibiting the formation and accelerating the decay of convertase. CD55 prevents activation of the complement system and increased expression level of CD55 can inhibit progression of myopia.

Microorganisms in different organs/tissues are different and always changing. According to research available now, composition and balance of microorganisms play an important role in our health and diseases. In autoimmune uveitis model, Horai et al. (2015) found that intestinal microorganisms induce autoreactive T helper 17 (Th17) cells to induce retina-involved uveitis. The use of antibiotics can relieve the uveitis and reduce the amount of autoreactive Th17 cells. Broad-spectrum antibiotic supplementation can not only up-regulate regulatory T cells but also suppress autoreactive effector T cells. It is also found that human leukocyte antigen B27 is affecting immune system by increasing intestinal permeability and associated with autoimmune uveitis and gut dysbiosis. Moreover, gut dysbiosis is one of the factors involved in progression of type II diabetes mellitus (DM), which causes concentrations of lipids, fatty acids, and glucose in plasma to increase and changes/activates immune cells to secret inflammatory molecules (TNFα, IL-1β, IL-6, IFN-γ, inflammatory adipokines, and chemokines). These factors also affect diabetic retinopathy (DR). Chronic low-grade inflammation related to metabolic disorder and oxidative stress is also associated with changes in gut microbiota composition. The gut microbiota is linked to age-related macular degeneration (AMD). Compared with healthy people, AMD patients at late stage also have gut dysbiosis.

The mammalian intestine harbors a plurality of symbiotic bacteria (commensal microbiota) which have interactions with their host through secreted proteins or metabolites and bacterial membrane vesicles (BMV). BMVs are produced at different stages of bacterial growth. Thus BMV is considered as a long-distance communication and signaling system and involved in interactions between host and bacteria or between bacteria. The membrane vesicles released from the outer membranes of Gram-positive bacteria are called Outer membrane vesicles (OMVs) while Gram-positive bacteria release membrane vesicles (MV). The research now indicates that BMV is associated with microbial pathogenesis and diseases. In the presence of antibiotics, BMVs increase the survival of bacteria and deliver antibiotic-resistant genes or antibiotic degrading enzyme to other bacterial and thus cause diseases. BMVs send microbe-associated molecular patterns (MAMPs) to host cells and this may induce inflammation or anti-inflammatory response. Generally, BMVs derived from pathogenic bacteria have pro-inflammatory effects on the host cell while probiotic BMVs have immunomodulatory activity. Moreover, BMVs can deliver DNA or RNA into cells to induce cellular protective mechanism through pattern recognition receptors or regulation of gene expression of the host cells. OMVs can be detected in cerebral spinal fluid and this represents that OMVs can pass the blood-brain barrier. Metagenome analysis of BMV can be used to determine the interactions between bacteria and the host. By metagenome analysis of serum extracellular vesicles, it is found that patients with psoriasis or asthma have different microbiota.

### SUMMARY

Therefore, it is a primary object of the present invention to provide a Lactiplantibacillus plantarum subsp. plantarum strain EP21 which is used for improvement of eye diseases and deposited in food industry research and development institute (FIRDI) with an accession number of BCRC911210.

Preferably, the Lactiplantibacillus plantarum subsp. plantarum strain EP21 includes a segment of sequence shown in Seq ID No. 3.

It is another object of the present invention to provide a Lactiplantibacillus plantarum subsp. plantarum strain EP21 used to prepare a pharmaceutical composition for improvement of eye diseases.

Preferably, the pharmaceutical composition includes a pharmaceutically acceptable excipient, carrier, adjuvant and/or food additive.

Preferably, the pharmaceutically acceptable carrier includes, but not limited to, solvent, buffer, emulsifier, suspending agent, decomposer, disintegrating agent, dispersing agent, binding agent, excipient, stabilizing agent, chelating agent, diluent, gelling agent, preservative, wetting agent, lubricant, absorption delaying agent, and liposome and its analogues and substitutes. Persons skilled in the art can select suitable excipients and adjust ratios according to their professional or routine techniques.

Preferably, the carrier suitable for the present invention includes, but not limited to water, normal saline, phosphate buffered saline (PBS), aqueous solution containing alcohol, and a combination thereof.

Preferably, the food additives are substances added to edible materials for preparing products that animals (including human being) can eat. A use and a ratio of the food additives are determined by persons skilled in the art according to their professional or routine techniques. The food additives include natural and artificial sweeteners, coloring agents, curing and pickling agent, flavor agent, emulsifier, fat substitute, hardening agent, leavening agent, lubricant, moisturizer, preservative, stabilizer, and thickener.

Preferably, a dosage form of the pharmaceutical composition includes, but not limited to, solution, suspension, capsule, pill, lozenge, troche, powder, freeze-dried powder, lotions, emulsions, suppository, slurry, and ointment.

More preferably, using techniques learned by persons skilled in the art, an isolated strain of Lactiplantibacillus plantarum subsp. plantarum or its subcultures is/are prepared together with a pharmaceutically acceptable vehicle to make up a dosage form suitable for oral administration.

Preferably, an effective dose of the pharmaceutical composition is application of 1×10⁶ cfu/kg to 1×10¹⁰ cfu/kg to recipients per day.

More preferably, an effective dose of the pharmaceutical composition is application of 5×10⁸ cfu/kg to 5×10⁹ cfu/kg to recipients per day.

Preferably, the eye diseases include, but not limited to, myopia, dry eye syndrome (DES), eye fatigue, keratitis, macular degeneration, retinitis pigmentosa (RP), proliferative diabetic retinopathy (PDR), ischemic retinopathy, choroidal neovascularization, glaucoma, systemic lupus erythematosus (SLE), and Sjögren's syndrome (SS).

More preferably, the ischemic retinopathy includes diabetic retinopathy (DR), retinopathy of prematurity (ROP), central retinal vein occlusion (CRVO), and ischemic optic neuropathy (ION).

It is a further object of the present invention to provide a composition containing bacterial membrane vesicles for improvement of eye diseases. The bacterial membrane vesicles (MVs) are derived from the Lactiplantibacillus plantarum subsp. plantarum strain EP21 mentioned above.

Preferably, a vesicle diameter of the MVs is ranging from 100 nm to 300 nm.

Preferably, a vesicle diameter of the MVs is ranging from 120 nm to 255 nm.

It is a further object of the present invention to provide a use of a pharmaceutical composition containing the membrane vesicles (MVs) mentioned above for improvement of eye diseases.

After applying the present Lactiplantibacillus plantarum subsp. plantarum strain EP21 and the MVs of the Lactiplantibacillus plantarum subsp. plantarum strain EP21 to recipients, the eye diseases can be improved by inhibition of excessive increase in the axial length and changes in the refraction of eyes. The expressions of activated p-NFκB, NFκB, NLRP3, IL1β, TNFα, and IL6 are also inhibited while expression of anti-inflammatory target IL10 is increased.

Preferably, the pharmaceutical composition containing the membrane vesicles (MVs) is prepared into dosage forms suitable for injections or putting in eye drops to be applied to recipients. Types of the injections include intravenous injections (IV), intraperitoneal injections (IP), and tail vein injections.

Preferably, an effective dose of the pharmaceutical composition containing the MVs is application of 1×10⁵ particles/kg to 1×10¹¹ particles/kg to recipients per day.

More preferably, the optimal effective dose of the pharmaceutical composition containing the MVs is application of 2.7×10⁸ particles/kg to 5.3×10⁹ particles/kg to recipients per day.

The followings are detailed descriptions of methods for preparation of a composition containing Lactiplantibacillus plantarum subsp. plantarum strain EP21 and membrane vesicles (MVs) derived from Lactiplantibacillus plantarum subsp. plantarum strain EP21, tests of pharmaceutical compositions prepared by Lactiplantibacillus plantarum subsp. plantarum strain EP21 or its membrane vesicles (MVs) for improvement of eye diseases, and tests and analysis of inhibition effects of the pharmaceutical compositions prepared by Lactiplantibacillus plantarum subsp. plantarum strain EP21 or its membrane vesicles (MVs) on inflammatory factors. Thereby it is approved that the composition containing Lactiplantibacillus plantarum subsp. plantarum strain EP21 or its membrane vesicles (MVs) do improve eye diseases. Moreover, the membrane vesicles (MVs) and its pharmaceutical composition can pass the blood retinal barrier (BRB) through blood flow, enhance immune regulatory properties of cells, and alleviate retinal inflammation induced by TGF-β2 and thus further slow down myopia progression induced by TGF-β2.

### BRIEF DESCRIPTION OF THE DRAWINGS

FIG. 1A is a bar chart of rat axial length of an embodiment (embodiment one) in which NC is a negative control group (injected with balanced salt solution, eye wash buffer), EP21 is a treatment group fed with EP21 by oral gavage, T2 is a positive control group (injected with TGF-β2 and fed with 1×PBS by oral gavage for 3 weeks), and T2+EP21 is a group induced with TGF-β2 and treated with EP21 according to the present invention;

FIG. 1B is a bar chart of relative changes in rat refraction (R eye/L eye) of the embodiment one in which NC is a negative control group (injected with balanced salt solution, eye wash buffer), EP21 is a treatment group fed with EP21 by oral gavage, T2 is a positive control group (injected with TGF-β2 and fed with 1×PBS by oral gavage for 3 weeks), and T2+EP21 is a group induced with TGF-β2 and treated with EP21 according to the present invention;

FIG. 2A is an immunofluorescence (IF) picture of TGF-β of the embodiment one in which NC is a negative control group (injected with balanced salt solution, eye wash buffer), EP21 is a treatment group fed with EP21 by oral gavage, T2 is a positive control group (injected with TGF-β2 and fed with 1×PBS by oral gavage for 3 weeks), and T2+EP21 is a group induced with TGF-β2 and treated with EP21 according to the present invention (scale bar: 165 μm);

FIG. 2B is a bar chart showing quantification of IF intensity of the groups in FIG. 2A;

FIG. 3A is an immunofluorescence (IF) picture of MMP2 of the embodiment one in which NC is a negative control group (injected with balanced salt solution, eye wash buffer), EP21 is a treatment group fed with EP21 by oral gavage, T2 is a positive control group (injected with TGF-β2 and fed with 1×PBS by oral gavage for 3 weeks), and T2+EP21 is a group induced with TGF-β2 and treated with EP21 according to the present invention (scale bar: 165 μm);

FIG. 3B is a bar chart showing quantification of IF intensity of the groups in FIG. 3A;

FIG. 4A is an immunofluorescence (IF) picture of COL1A1 of the embodiment one in which NC is a negative control group (injected with balanced salt solution, eye wash buffer), EP21 is a treatment group fed with EP21 by oral gavage, T2 is a positive control group (injected with TGF-β2 and fed with 1×PBS by oral gavage for 3 weeks), and T2+EP21 is a group induced with TGF-β2 and treated with EP21 according to the present invention (scale bar: 165 μm);

FIG. 4B is a bar chart showing quantification of IF intensity of the groups in FIG. 4A;

FIG. 5A is an immunofluorescence (IF) picture of NFκB of the embodiment one in which NC is a negative control group (injected with balanced salt solution, eye wash buffer), EP21 is a treatment group fed with EP21 by oral gavage, T2 is a positive control group (injected with TGF-β2 and fed with 1×PBS by oral gavage for 3 weeks), and T2+EP21 is a group induced with TGF-β2 and treated with EP21 according to the present invention (scale bar: 165 μm);

FIG. 5B is a bar chart showing quantification of IF intensity of the groups in FIG. 5A;

FIG. 6A is an immunofluorescence (IF) picture of TNF-α of the embodiment one in which NC is a negative control group (injected with balanced salt solution, eye wash buffer), EP21 is a treatment group fed with EP21 by oral gavage, T2 is a positive control group (injected with TGF-β2 and fed with 1×PBS by oral gavage for 3 weeks), and T2+EP21 is a group induced with TGF-β2 and treated with EP21 according to the present invention (scale bar: 165 μm);

FIG. 6B is a bar chart showing quantification of IF intensity of the groups in FIG. 6A;

FIG. 7A is a western blotting analysis of NFκB of the embodiment one in which NC is a negative control group (injected with balanced salt solution, eye wash buffer), T2 is a positive control group (injected with TGF-β2 and fed with 1×PBS by oral gavage for 3 weeks), and T2+EP21 is a group induced with TGF-β2 and treated with EP21 according to the present invention;

FIG. 7B is a bar chart showing quantitative results of the groups in FIG. 7A;

FIG. 8 is a bar chart showing cell viability of another embodiment (embodiment two) in which the vertical ordinate represents different numbers (8×10⁸, 4×10⁸, 2×10⁸, and 1×10) of EP21 MVs used to treat retinal pigmented epithelial cells RPE1 according to the present invention;

FIG. 9A is a western blotting analysis of RPE1 cells of a further embodiment (embodiment three) in which NC is a control group, IL-1β is a group treated with 1.25 ng/ml IL-1β, and IL-1β+MV is a group pretreated with 2.2×10⁶ EP21 MVs and then added with IL-1β according to the present invention;

FIG. 9B is a bar chart showing quantitative results of the groups in FIG. 9A;

FIG. 9C is a western blotting analysis of ARPE19 cells of the embodiment three in which NC is a control group, IL-1β is a group treated with 1.25 ng/ml IL-1β, and IL-1β+MV is a group pretreated with 2.2×10⁶ EP21 MVs and then added with IL-1β according to the present invention;

FIG. 9D is a bar chart showing quantitative results of the groups in FIG. 9C;

FIG. 9E is a western blotting analysis of hRPEpiC cells of the embodiment three in which NC is a control group, IL-1β is a group treated with 1.25 ng/ml IL-1B, and IL-1B+MV is a group pretreated with 2.2×10⁶ EP21 MVs and then added with IL-1β according to the present invention;

FIG. 9F is a bar chart showing quantitative results of the groups in FIG. 9E;

FIG. 10A is a western blotting analysis of RPE1 cells of a further embodiment (embodiment four) in which NC is a control group, IL-1β is a group treated with 1.25 ng/ml IL-1β, IL-1β+MV is a group applied with IL-1β and MVs, IL-1β+MV+DNase I is a group applied with IL-1β, MVs and DNase I, IL-1B+MV+RNase A is a group applied with IL-1β, MVs, and RNase A according to the present invention while MV(s) means 2.2×10⁶ EP21 membrane vesicles and final concentration of both DNase I and RNase A is 100 ng;

FIG. 10B is a bar chart showing quantitative results of the groups in FIG. 10A;

FIG. 11A is a bar chart showing expression of IL6 of a further embodiment (embodiment five) in which cells are respectively applied with TNFα (2.5 ng/ml), and TNFα+MVs of strain EP21 according to the present invention;

FIG. 11B is a bar chart showing expression of IL6 of the embodiment five in which cells are respectively applied with IL1B (1.25 ng/ml) and IL1B+MVs of strain EP21 according to the present invention;

FIG. 12 is a bar chart showing expression levels of IL6 determined using enzyme-linked immunosorbent assay (ELISA) of the embodiment five in which NC is a control group, TNFα is a group applied with 2.5 ng/ml TNFα, TNFα+EP21 RNA is a group applied with TNFα+EP21 RNA (1.5 μg), TNFα+EP21 MV RNA is a group applied with TNFα+EP21 MV RNA (1.5 μg) according to the present invention;

FIG. 13A is a bar chart of rat axial length of a further embodiment (embodiment six) in which NC is a negative control group (injected with balanced salt solution, eye wash buffer), T2 is a positive control group (injected with TGF-β2 and put in eye drops 1×PBS for 3 weeks), T2+EP21 MV (3.3×10⁹) ED is a group applied with eye drops containing 3.3×10⁹ MV particles of strain EP21 for 3 weeks, and T2+EP21 MV (9.9×10⁸) ED is a group applied with eye drops containing 9.9×10⁸ MV particles of strain EP21 for 3 weeks;

FIG. 13B is a bar chart of relative changes (R eye/L eye) in rat refraction of the embodiment six in which NC is a negative control group (injected with balanced salt solution, eye wash buffer), T2 is a positive control group (injected with TGF-β2 and put in eye drops 1×PBS for 3 weeks), T2+EP21 MV (3.3×10⁹) ED is a group applied with eye drops containing 3.3×10⁹ MV particles of strain EP21 for 3 weeks, and T2+EP21 MV (9.9×10⁸) ED is a group applied with eye drops containing 9.9×10⁸ MV particles of strain EP21 for 3 weeks;

FIG. 14A is a bar chart of rat axial length of a further embodiment (embodiment seven) in which NC is a negative control group (injected with balanced salt solution, eye wash buffer) injected with 1×PBS via tail vein injection, T2 is a positive control group (injected with TGF-β2 and injected with 1×PBS via tail vein injection for 3 weeks), T2+EP21 MV (3.3×10⁹) IV is a group injected with 1×PBS containing 3.3×10⁹ MV particles of strain EP21 via tail vein injection for 3 weeks, and T2+EP21 MV (3.3×10⁸) IV is a group injected with 1×PBS containing 3.3×10⁸ MV particles of strain EP21 via tail vein injection for 3 weeks;

FIG. 14B is a bar chart of relative changes (R eye/L eye) in rat refraction of the embodiment six in which NC is a negative control group (injected with balanced salt solution, eye wash buffer) injected with 1×PBS via tail vein injection, T2 is a positive control group (injected with TGF-β2 and injected with 1×PBS via tail vein injection for 3 weeks), T2+EP21 MV (3.3×10⁹) IV is a group injected with 1×PBS containing 3.3×10⁹ MV particles of strain EP21 via tail vein injection for 3 weeks, and T2+EP21 MV (3.3×10⁸) IV is a group injected with 1×PBS containing 3.3×10⁸ MV particles of strain EP21 via tail vein injection for 3 weeks;

FIG. 15A is a bar chart of rat axial length of the embodiment seven in which NC is a negative control group (injected with balanced salt solution, eye wash buffer) injected with 1×PBS via intraperitoneal injection, T2 is a positive control group (injected with TGF-β2 and injected with 1×PBS via intraperitoneal injection for 3 weeks), T2+EP21 MV (3.3×10⁹) IP is a group injected with 1×PBS containing 3.3×10⁹ MV particles of strain EP21 via intraperitoneal injection for 3 weeks, and T2+EP21 MV (3.3×10⁸) IP is a group injected with 1×PBS containing 3.3×10⁸ MV particles of strain EP21 via intraperitoneal injection for 3 weeks;

FIG. 15B is a bar chart of relative changes (R eye/L eye) in rat refraction of the embodiment seven in which NC is a negative control group (injected with balanced salt solution, eye wash buffer) injected with 1×PBS via intraperitoneal injection, T2 is a positive control group (injected with TGF-β2 and injected with 1×PBS via intraperitoneal injection for 3 weeks), T2+EP21 MV (3.3×10⁹) IP is a group injected with 1×PBS containing 3.3×10⁹ MV particles of strain EP21 via intraperitoneal injection for 3 weeks, and T2+EP21 MV (3.3×10⁸) IP is a group injected with 1×PBS containing 3.3×10⁸ MV particles of strain EP21 via intraperitoneal injection for 3 weeks;

FIG. 15C is a western blotting analysis of the embodiment seven in which NC represents a negative control group (injected with balanced salt solution, eye wash buffer), T2 is a positive control group (injected with TGF-β2 and injected with 1×PBS for 3 weeks), T2+MV (3.3×10⁹) is a group injected with 1×PBS containing 3.3×10⁹ MV particles of strain EP21 for 3 weeks;

FIG. 16A is an immunofluorescence (IF) picture of the embodiment seven in which isotype control and NC are negative control groups (injected with balanced salt solution, eye wash buffer), T2 is a positive control group (injected with TGF-β2 and injected with 1×PBS for 3 weeks), and T2+MV is a group induced with TGF-β2 and treated with 3.3×10⁹ MV particles of strain EP21 according to the present invention (scale bar: 165 μm);

FIG. 16B is an immunofluorescence (IF) picture of NFκB of the embodiment seven in which NC is a negative control groups (injected with balanced salt solution, eye wash buffer), T2 is a positive control group (injected with TGF-β2 and injected with 1×PBS for 3 weeks), and T2+MV is a group induced with TGF-β2 and treated with 3.3×10⁹ MV particles of strain EP21 according to the present invention (scale bar: 165 μm);

FIG. 16C is a bar chart showing quantification of IF intensity of the groups in FIG. 16B;

FIG. 16D is an immunofluorescence (IF) picture of IL10 of the embodiment seven in which NC is a negative control groups (injected with balanced salt solution, eye wash buffer), T2 is a positive control group (injected with TGF-β2 and injected with 1×PBS for 3 weeks), and T2+MV is a group induced with TGF-β2 and treated with 3.3×10⁹ MV particles of strain EP21 according to the present invention (scale bar: 165 μm);

FIG. 16E is a bar chart showing quantification of IF intensity of the groups in FIG. 16D;

FIG. 16F is an immunofluorescence (IF) picture of TNFα of the embodiment seven in which NC is a negative control groups (injected with balanced salt solution, eye wash buffer), T2 is a positive control group (injected with TGF-β2 and injected with 1×PBS for 3 weeks), and T2+MV is a group induced with TGF-β2 and treated with 3.3×10⁹ MV particles of strain EP21 according to the present invention (scale bar: 165 μm);

FIG. 16G is a bar chart showing quantification of IF intensity of the groups in FIG. 16F;

FIG. 16H is an immunofluorescence (IF) picture of NLRP3 of the embodiment seven in which NC is a negative control groups (injected with balanced salt solution, eye wash buffer), T2 is a positive control group (injected with TGF-β2 and injected with 1×PBS for 3 weeks), and T2+MV is a group induced with TGF-β2 and treated with 3.3×10⁹ MV particles of strain EP21 according to the present invention (scale bar: 165 μm);

FIG. 16I is a bar chart showing quantification of IF intensity of the groups in FIG. 16H;

FIG. 16J is an immunofluorescence (IF) picture of IL1β of the embodiment seven in which NC is a negative control groups (injected with balanced salt solution, eye wash buffer), T2 is a positive control group (injected with TGF-β2 and injected with 1×PBS for 3 weeks), and T2+MV is a group induced with TGF-β2 and treated with 3.3×10⁹ MV particles of strain EP21 according to the present invention (scale bar: 165 μm);

FIG. 16K is a bar chart showing quantification of IF intensity of the groups in FIG. 16J;

FIG. 17 is an immunofluorescence (IF) picture of an embodiment eight showing MVs of strain EP21 are engulfed by retinal pigmented epithelial cells RPE1 labelled with DIL according to the present invention;

FIG. 18A is an immunofluorescence (IF) picture of an embodiment nine showing MVs of strain EP21 pass blood-retinal barrier (BRB) through blood flows according to the present invention;

FIG. 18B is a bar chart showing quantification of IF intensity of the groups in FIG. 18A;

FIG. 19A is showing results of rat axial length of an embodiment ten in which T2 is a positive control group (injected with TGF-β2 and fed with 1×PBS using tubes for 3 weeks) and the rest three strains are Lactobacillus casei Shirota, Lactiplantibacillus plantarum subsp. plantarum PS128, and Lactobacillus paracasei NTU 101 according to the present invention;

FIG. 19B is immunohistochemistry (IHC) image of the embodiment ten (scale bar: 40 μm) according to the present invention;

FIG. 19C is a western blotting analysis of the embodiment ten in which T2 is a positive control group (injected with TGF-β2 and fed with 1×PBS by oral gavage for 3 weeks) and the rest four groups are respectively applied with Lactobacillus casei Shirota, Lactobacillus paracasei NTU 101, Lactiplantibacillus plantarum subsp. plantarum PS128, and the strain EP21 after injection of TGF-β2 according to the present invention;

FIG. 19D is a bar chart showing quantification of IF intensity of the groups in FIG. 19C.

The present strain EP21 has been deposited in China Center for Type Culture Collection. (CCTCC) with an accession number of CCTCC M 20242188.

### DETAILED DESCRIPTION

The following embodiments are used for further explanation of the present invention, not intended to limit the content of the present invention. People skilled in the art can make certain improvements and modifications, without departing from the scope of the present invention. Unless otherwise stated, the following terms used in the specification and respective claims have the definitions given below.

Please note that the singular expression “a” used in the description and the claims is intended to cover one or more items, such as at least one, at least two, or at least three, not only a single one. Moreover, the terms “including” and “having” used in the specifications and claims of the present invention are open-ended and does not of exclude additional, unrequired elements or components described in the specifications and claims. It should also be noted that the term “or” generally also includes “and/or” unless the content clearly indicates otherwise. The terms “about” or “substantially” used in the specification and patent scope of this application are used to modify any error that may vary slightly, but such slight variations will not change nature of the present invention.

Probiotics or probiotic bacteria are microorganisms whose cells, mixed strains, extracts, or metabolites have beneficial effects on host's health. They are usually originated from human body or from supplements and beneficial to people.

The “pre-test measurement” mentioned in the specification means measurement of axial length and refraction of eyes of respective experiment animals. The pre-test measurement includes the following steps. Apply 50-80 mg/kg liquid anesthetic agent (Zoletil®-50) to the test animal by Intraperitoneal injection and carry out chemical immobilization. Measure the fraction and the axial length respectively using a handheld strip retinal detector and A-scan ultrasound biometry. According to the measurement results, it is expected that differences in the axial lengths and the refraction between two eyes are respectively no more than 0.02 mm and 2(D). Mice with innate eye detects found by the pre-test measurement may cause errors in experiment results and thus will not be counted in statistics at the following experiments.

The flow chart and steps of the respective embodiments comply with laboratory safety guidelines. The term “effective dose” mentioned in the specification refers to dosage range obtained according to experiment data and results of respective embodiment of the present invention. Persons skilled in the art can select suitable receptors according to their professional or routine techniques and adjust dose ratio and convert the dose according to types of recipients and dosage forms applied.

### Preparation Embodiment 1: Sources and Preparation of Test Strain

The bacteria strain according to the present invention is obtained and isolated from stool of Hamsters fed with Chinese medicines. The strain is inoculated on a De Man, Rogosa and Sharpe (MRS) agar plate (BD) using a four quadrant streak method and cultured for 16-18 hrs. Then bacterial liquid is diluted 100× and cultured in 40 ml MRS at 37° C. for 16-18 hrs. Next bacterial liquid obtained is diluted 100× and cultured in 1000 ml MRS at 37° C. for 24 hrs. Select a single colony of probiotics on the plate and amplify a part of sequence of its 16S rRNA gene by a specific primer pair for bacteria such as SEQ ID NO: 1 (forward primer: 8F primer) and SEQ ID NO: 2 (reverse primer: 1492R primer) to get nucleic acid segment such as sequence shown in SEQ ID NO: 3. Methods for total RNA extraction are learned by persons skilled in the art and there is no more detailed description. Next compare sequence of the nucleic acid segment (SEQ ID NO: 3) with six 16S rRNA gene sequences of Lactiplantibacillus plantarum subsp. plantarum (accession no. KX057658.1, ON506095.1, MK524162.1, MT613641.1, MT597711.1, and MT597692.1) at GenBank of National Center for Biotechnology Information (NCBI), the sequences show 99% similarity. Thus the strain of the present invention is Lactiplantibacillus plantarum subsp. plantarum (L. plantarum), hereafter abbreviated as strain EP21.

The following are physiological properties of the strain EP21: growth temperature ranging from 35° C. to 40° C., growth pH 4.0-7.0, oxygen requirement level: facultative anaerobic. The present strain EP21 gives a positive result in the Gram stain test.

The present strain EP21 has been deposited in Biosource Collection and Research Center (BCRC) (331 Shih-Pin Rd., Hsinchu 300, Taiwan) of food industry research and development institute (FIRDI) with an accession number of BCRC 911210 on Nov. 21, 2023.

### Preparation Embodiment 2: Extraction of Membrane Vesicles (MV)

The bacterial liquid of the strain EP21 (abbreviated as EP21 hereafter) is centrifuged at 8000 rpm for 1 hour and collect supernatant. Filter the supernatant with 0.22 μm filter cup and concentrate with centrifugal filter to 10× volume. Place into an ultracentrifuge for centrifugation at 28000 rpm, 4° C. for 6 hrs and collet pellet. Then dissolve the pellet with 50% OptiPrep™ solution and add 45%, 40%, 35%, 30%, 25%, and 20% OptiPrep™ solution into ultracentrifuge tubes in sequence to perform density gradient centrifugation (28000 rpm, 4° C., overnight) for density gradient separation. The isolated EP21 membrane vesicles (MVs) are stored at −80° C. After preparation, use transmission electron microscope (JEM 1400 FLASH) to detect morphology of the MV. After negative stain of EP21 MVs using 1% PTA (phosphotungstic acid and applied to 400 mesh copper grids (Model: 01754-F, TED PELLA) for 1 minute, remove excess solution by filter paper and place into dryer before observation. The transmission electron microscope applies 120 KV to accelerate a beam of electrons which is transmit through the MV. Thus different fractions after density gradient separations (ODG fractions) and morphology of EP21 MV are observed. The result shows that a vesicle diameter of the present EP21 MV is about 177.95±32.6 nm.

Use nanoparticle tracking analysis (NTA) (version NTA 3.4 Build 3.4.003) to detect vesicle (particle) size and the number of vesicles of the EP21 MV. The instrument is set at SOP Standard Measurement for detection and results obtained is multiplied by dilution ratio to estimate the number of vesicles of the EP21 MV for applications in the following experiments. The NTA result shows that the vesicle diameter of the present EP21 MV is about 156.36±25.7 nm.

Liquid nitrogen is used for snap freezing of the prepared EP21 MV to fix its morphology on 0.2 μm PC film (Model: GTTP02500, Isopore™). After platinum coating on a surface, use Cryo Scanning Electron microscopy (JEOL JSM-7800F) to observe vesicle size and morphology of the EP21 MV. Use 5 Mm fluorescent dye DIL (CAS no: 41085-99-8, Alfa Aesar, Switzerland) for hydrophobic labelling of the purified EP21 MV (30 mins, 37° C.), remove unbound dye by ultracentrifugation (28000 rpm, 4° C. for 2 hrs), and freeze at −80° C. for the following phagocytosis of MVs and tracking of MVs in vivo experiments. The results of the Cryo Scanning Electron microscopy indicate that the vesicle diameter of the EP21 MV is about 153.04±17.6 nm and the vesicle diameter of the isolated EP21 MV is about 195.28±59.5 nm.

### Embodiment 1: Test of Feeding EP21 for Inhibition of Myopia

Use 3-week-old Brown Norway rat as test animals. After pre-test measurement of the animals, inject 250 ng/ml TGF-β2 (transforming growth factor-beta 2) to an upper eyelid of a right eye once a week to induce inflammation for 3 weeks. In a negative control group, inject balanced salt solution (BSS, eye wash buffer used tor preparing injection medicines) and feed with 1×PBS using tubes for 3 weeks. In a positive control group, inject TGF-β2 and feed with 1×PBS using tubes for 3 weeks. A treatment group is injected with TGF-β2 and having tube feeding with strain EP21 on exponential phase (2.5-3×10⁹ CFU/0.1 ml/day) for 3 weeks. Measure the axial length and the retraction before and after the treatment to verify changes among the respective groups. After taking whole eyeball as samples, perform western blotting, immunohistochemical staining, immunohistochemistry (IHC) analysis, and analysis of the amount of dopamine. Use INFINITE M NANO (TECAN) to measure growth curves for 24 hrs and select the strain EP21 on exponential phase to feed the animals. Pick up a colony of the strain EP21 and culture in 6 ml MRS (Model: 288130, BD) at 37° C. for 16-18 hrs. Each day dilute EP21 bacterial solution 100 times and culture in 40 ml MRS at 37° C. for 6 hrs, to the exponential phase. Then centrifuge the EP21 bacterial solution at 3500 rpm for 15 mins, wash EP21 pellet with 1×PBS. After centrifugation, dissolve with 1×PBS to form an EP21 and each rat is fed with 0.1 ml (2.5-3×10⁹ CFU/ml) of the EP21.

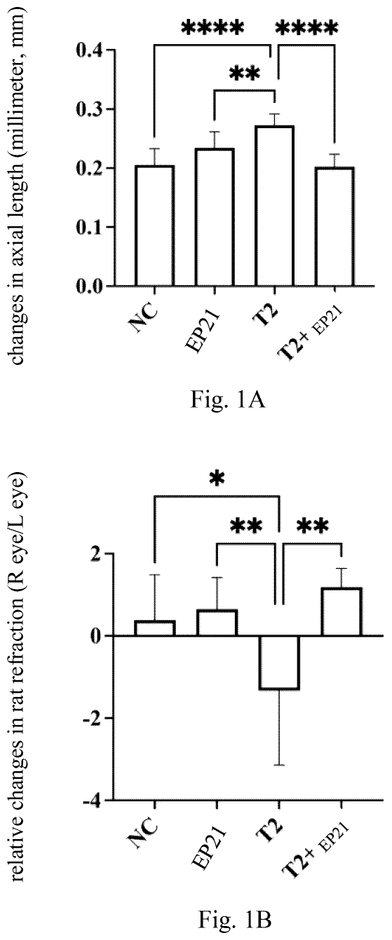

As the results shown in FIG. 1A and FIG. 1B, TGF-β2 significantly increases the axial length and changes the refraction of the rat. The feeding of the EP21 inhibits an excessive increase in the axial length and changes in the refraction. In this embodiment, one-way analysis of variance (ANOVA) is used as a statistical method. * means P value<0.05; means p-value<0.01; *** means p-value<0.001; **** means p-value<0.0001.

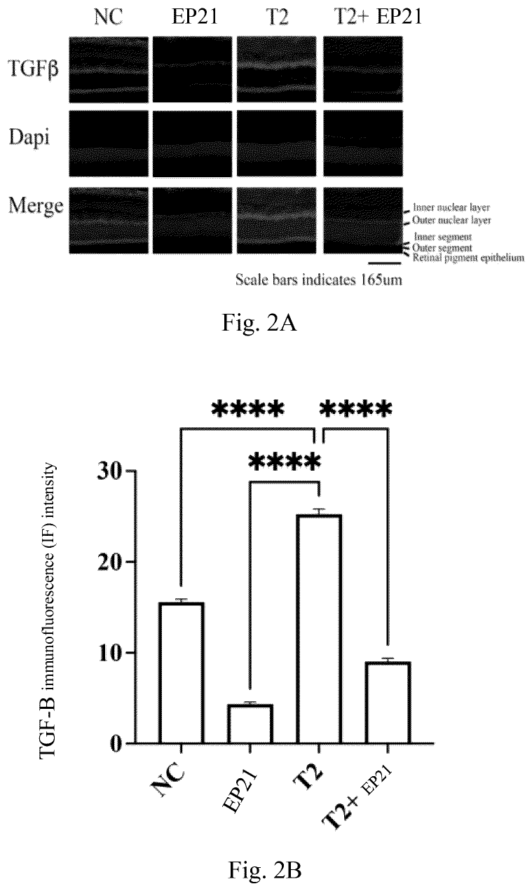

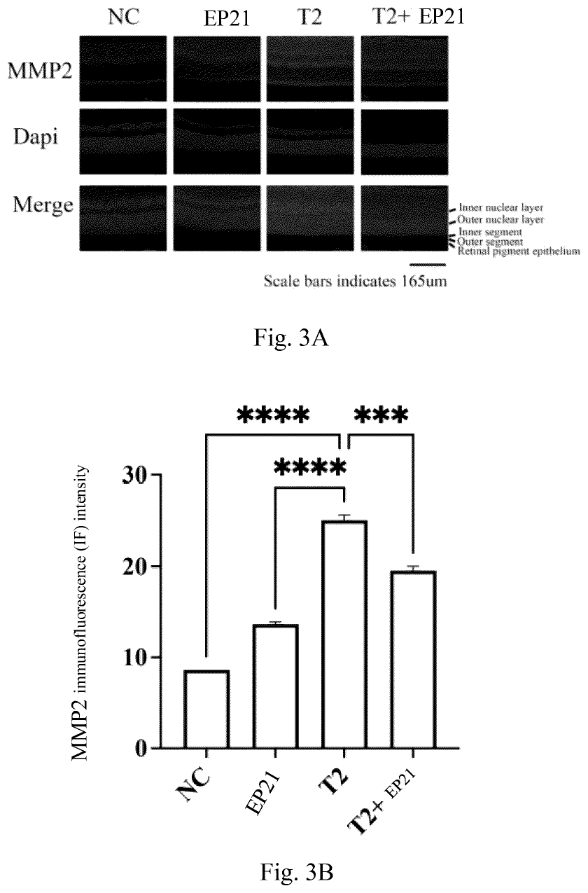

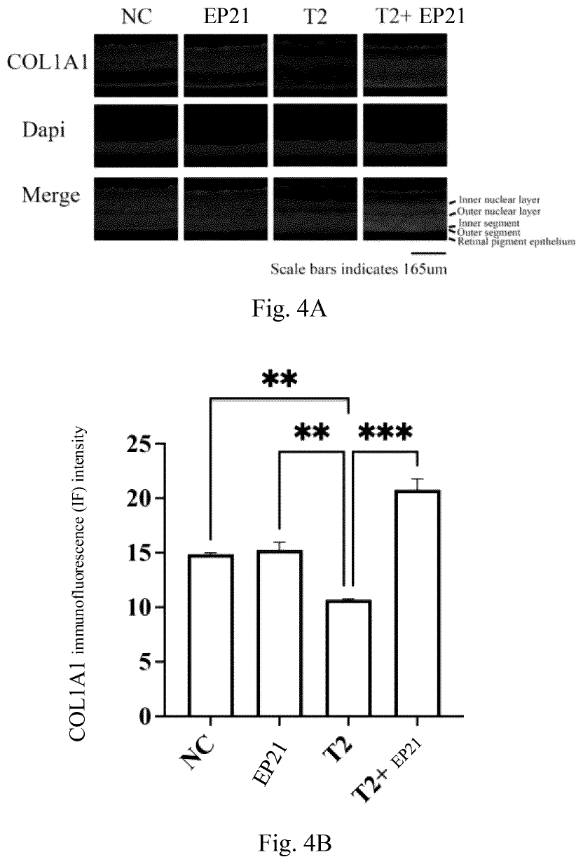

Use immunofluorescence (IF) to analyze effects of feeding the EP21 on TGF-β, MMP2 (matrix metalloproteinase-2), and COL1A1 (alpha-1 type I collagen) after using TGF-β2 to induce myopia in the eyeball. Fixed tissue is embedded in paraffin, deparaffinization after section, rehydration, and boiling in an epitope exposure reagent. Nonspecific binding sites on the tissue are blocked by BSA 1% at room temperature for 1 hr. After reaction with primary antibody at 4° C. overnight, react with secondary antibody at room temperature for 1 hr. Use DAPI (4′,6-diamidino-2-phenylindole) (1:1000) for nuclear staining, protected from light and react for 5 mins. After mounted with anti-bleaching reagent, use a fluorescence microscope for fluorescence excitation and capturing images. Perform quantitative analysis of experiment results by Image J and carry out One-way ANOVA analysis of numerical values in GraphPad Prism (statistical software). P value<0.05 (represent by *) is typically considered to be statistically significant. The results are shown in FIG. 2A-FIG. 4B. TGF-β2 increases respective expression of TGF-β, MMP2, and COL1A1 while the feeding of the EP21 inhibits TGF-β (FIG. 2A-FIG. 2B), MMP2 (FIG. 3A-FIG. 3B), and COL1A1 (FIG. 4A-FIG. 4B).

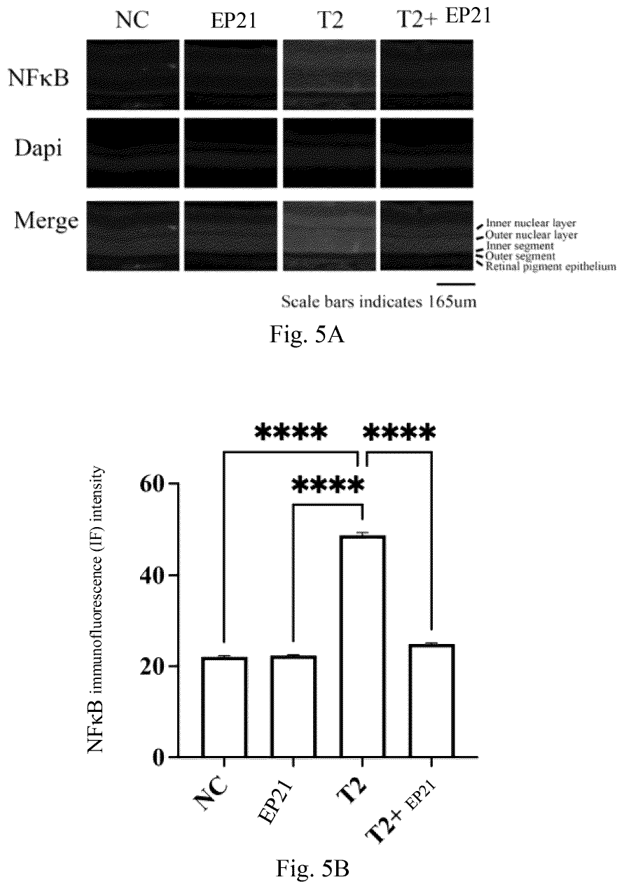

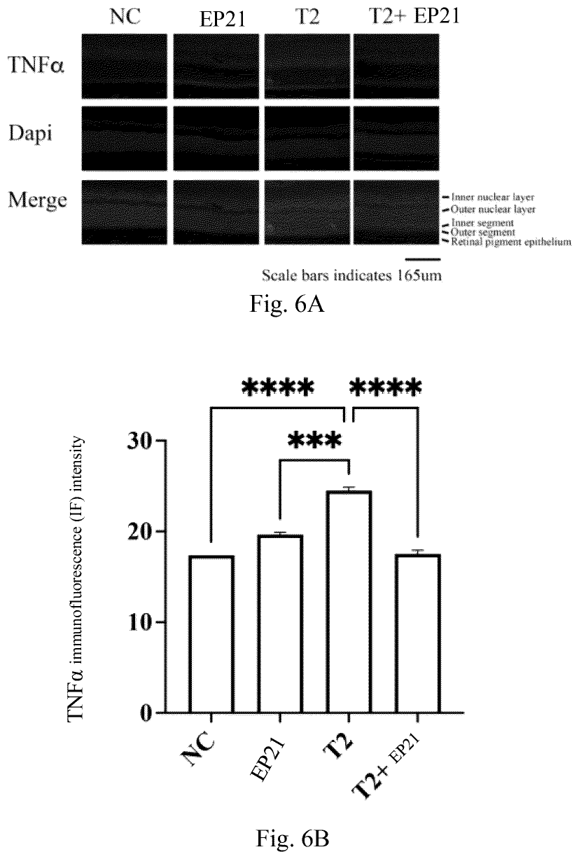

Use the above immunofluorescence to analyze expression of TGF-β2-induced transcription factor NFκB in the eyeball. Perform quantitative analysis of experiment results by Image J and carry out One-way ANOVA analysis of numerical values obtained in GraphPad Prism (statistics software). P value<0.05 is typically considered to be statistically significant. The results are shown in FIG. 5A-FIG. 6B. TGF-β2 enhances expression of NFκB and TNF-α and the feeding of the present EP21 inhibits NFκB (FIG. 5A-FIG. 5B) and TNF-α (FIG. 6A-FIG. 6B).

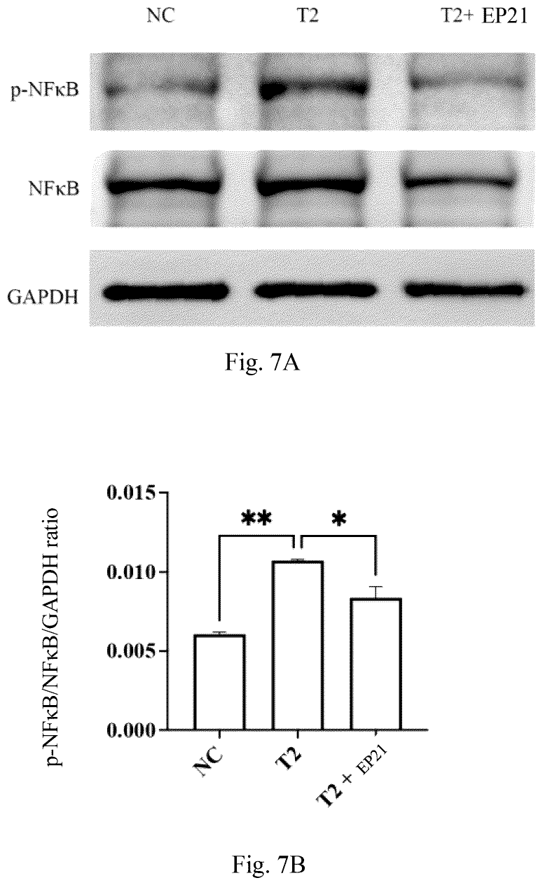

Next use western blotting to analyze expression amount of TGF-β2-induced transcription factor p-NFκB in the eyeball. Eyeball or retina tissue is added with Radio-Immunoprecipitation Assay (RIPA) lysis buffer (50 Mm Tris-HCl, 250 Mm NaCl, 1% NP-40, 0.5% Sodium deoxycholate, 0.1% Sodium dodecyl sulfate, protease inhibitor and phosphatase inhibitor) and cut into pieces. Extract proteins in the tissue by ultrasonic treatment. Then collect supernatant after centrifugation at 12000 rpm, 4° C. for 15 minutes. Carry out protein quantification of the samples using Bradford reagent (Bio-Rad, #5000006). Use 2 mg/ml BSA as a standard solution and linear range of a calibration line is 10 μg (microgram)-1 μg. SDS-PAGE (sodium dodecyl sulfate-polyacrylamide gel electrophoresis) is used for protein isolation. After electrophoresis, the protein samples are transferred from the gel to 0.45 μm polyvinylidene difluoride (PVDF) membrane for 50 minutes. The transferred membrane is treated by 5% skimmed milk at room temperature for 1 hour to fill and block the non-specific bonding sites on the membrane. After incubation with primary antibody at 4° C. overnight, add secondary antibody and react at room temperature for 1 hour. Next add enhanced chemiluminescence (ECL) reagent, use a luminometer to record results, and quantify the results by using Image J. Use the quantitative result of the target protein to calibrate its expression by internal standard method. GAPDH is used as a loading control antibody and quantitative analysis of experiment results by Image J is performed. Statistics on numerical values (p-NFκB/NFκB/GAPDH) obtained are performed and then One-way ANOVA analysis of the numerical values in GraphPad Prism is carried out. P value<0.05 is typically considered to be statistically significant. The results are shown in FIG. 7A-FIG. 7B. TGF-β2 activates expression of NFκB and the feeding of the present EP21 inhibits the activation of NFκB.

### Embodiment 2: Cell Viability Test

Seed retinal pigmented epithelial cells RPE1 into a 96-well plate at 3×10³ cells per well and culture about 16-18 hrs. After cell attachment, add different numbers of EP21 membrane vesicles (MVs) including 8×10⁸, 4×10⁸, 2×10⁸, and 1×10⁸ and treat for 24 hrs. React with cell viability reagent (20 μl PMS+2 ml MTS) for 2 hrs and measure absorbance at 490 nm. Survival rate is (A treat/A control)×100%, used to evaluate cytotoxicity of the EP21 MVs on the human retinal pigment epithelial cells RPE1.

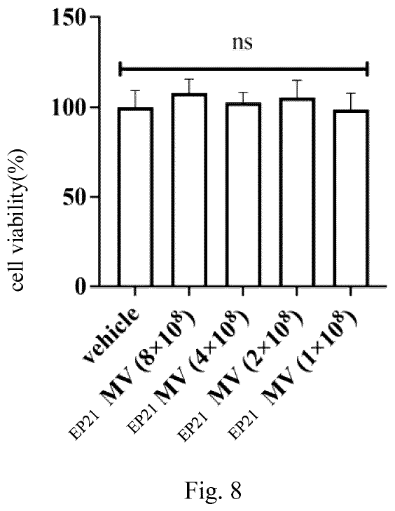

The EP21 MVs with different numbers of vesicles obtained in preparation embodiment 2 are used to treat the RPE1 cells for 24 hrs and cell growth is observed. Use cell viability reagent and GraphPad Prism to analyze results. The results are shown in FIG. 8. After treatment of EP21 MVs with different numbers of vesicles, cell viability of the RPE1 cells has no significant change (ns means no significant difference compared with the control group). Therefore, the present EP21 MVs show no cytotoxicity.

### Embodiment 3: Evaluation of Impact of EP21 MVs on Activation of Transcription Factor NFκB

Seed three kinds of human retinal pigment epithelial cells (RPE1, ARPE19 and hRPEpiC) respectively into a 6-well plate at 3×10⁵ cells per well and culture for about 16-18 hrs. Then use EP21 MVs (2.2×10⁶ vesicles/particles) for pretreatment and further treat with IL-1B (Interleukin-1 beta) (1.25 ng/ml) for 10 minutes.

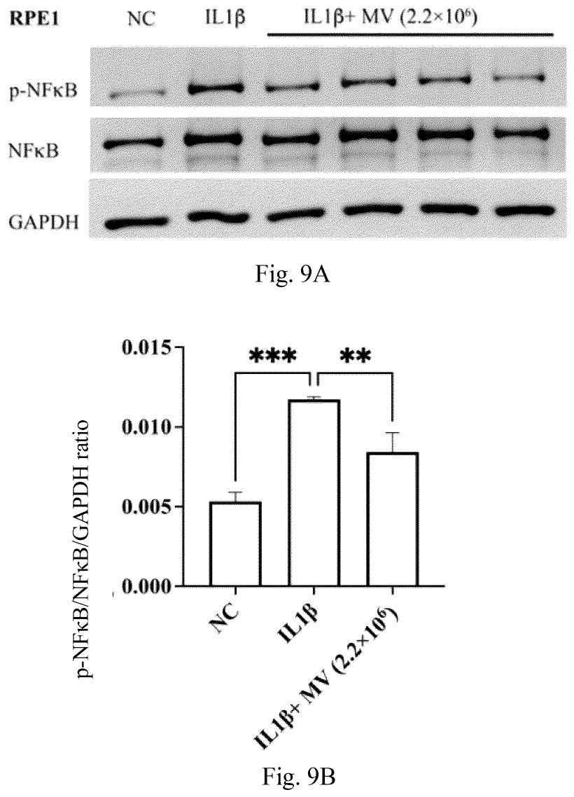

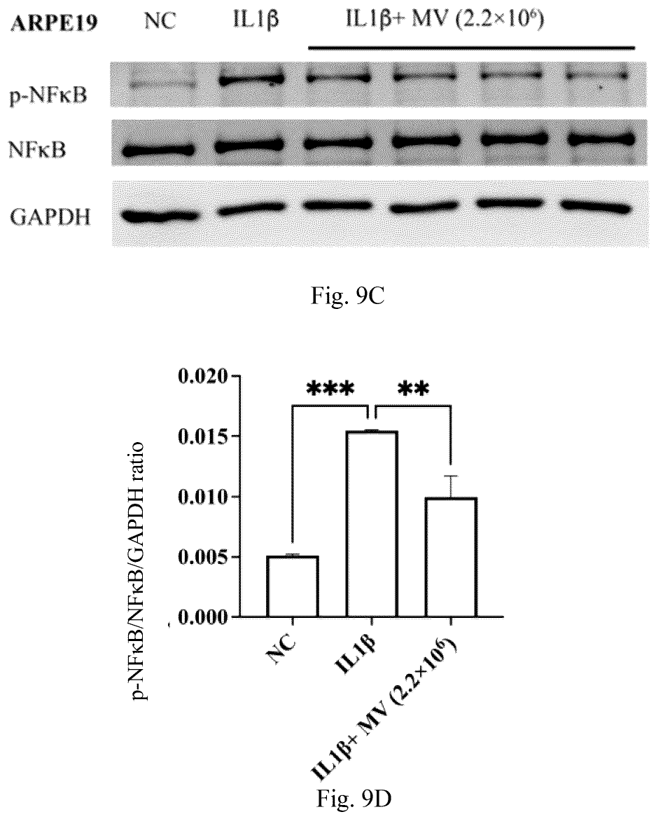

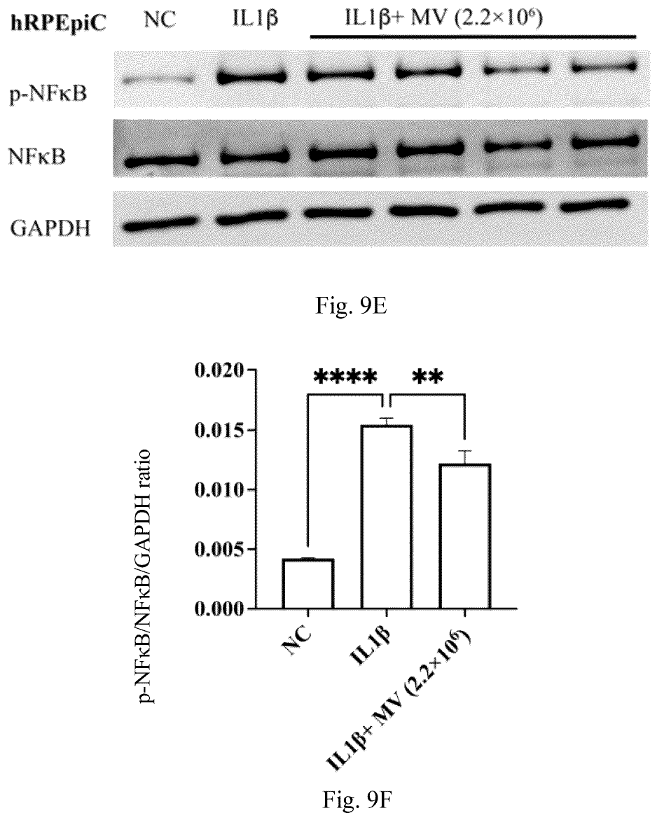

Use western blotting for analysis of expression amount of transcription factor p-NFκB. GAPDH is used as a loading control antibody and experiment results are quantified by Image J. Statistics on numerical values (p-NFκB/NFκB/GAPDH) obtained are performed and then One-way ANOVA analysis of the numerical values in GraphPad Prism is carried out. P value<0.05 is typically considered to be statistically significant. The results are shown in FIG. 9A-FIG. 9F. The groups treated with EP21 MVs all show that activation of IL-1ß-induced transcription factor NFκB is inhibited.

### Embodiment 4 Effect of Nucleases on EP21 MVs

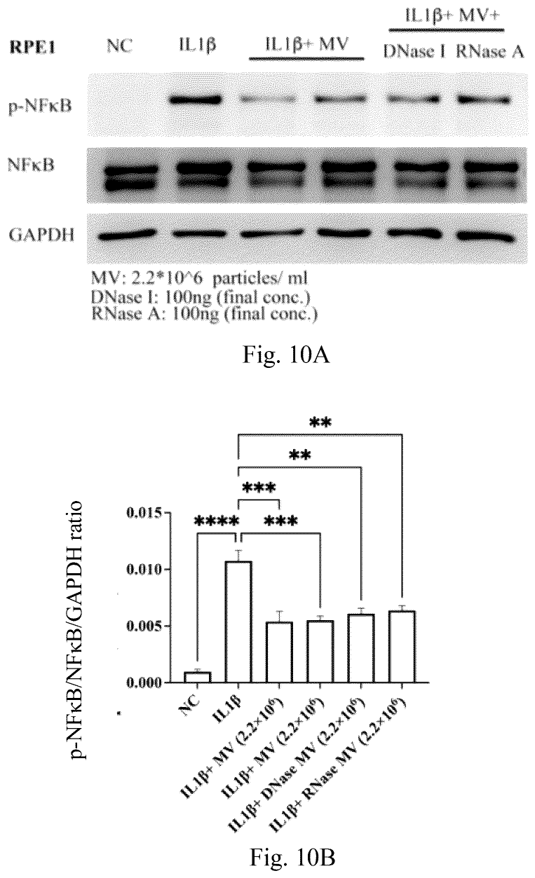

The EP21 MV treated by nucleases (DNase I and RNase A) with final concentration of 100 ng is added into human retinal pigmented epithelial cells RPE1 for pretreatment for 2 hrs. Then add inflammatory substance IL-1β(at a concentration of 1.25 ng/ml) to confirm that whether the EP21 MVs still provides anti-inflammatory ability to the human retinal pigmented epithelial cells after being treated with the nucleases. Use western blotting to analyze expression of transcription factor p-NFκB. GAPDH is used as a loading control antibody and experiment results are quantified by Image J. Statistics on numerical values (p-NFκB/NFκB/GAPDH) obtained are performed and then One-way ANOVA analysis of the numerical values in GraphPad Prism is carried out. P value<0.05 is typically considered to be statistically significant. The results shown in FIG. 10A-FIG. 10B indicate that activation of IL-1ß-induced transcription factor NFκB is inhibited in the groups treated with present EP21 MVs and the groups treated with nucleases.

### Embodiment 5: Cytokine Inhibition by EP21 MVs

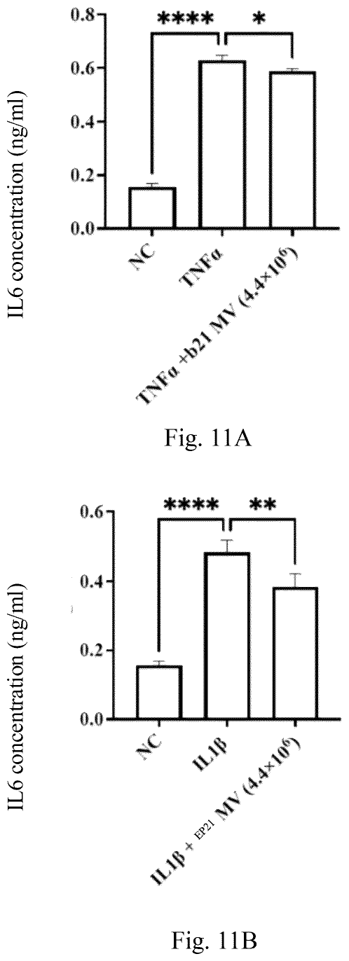

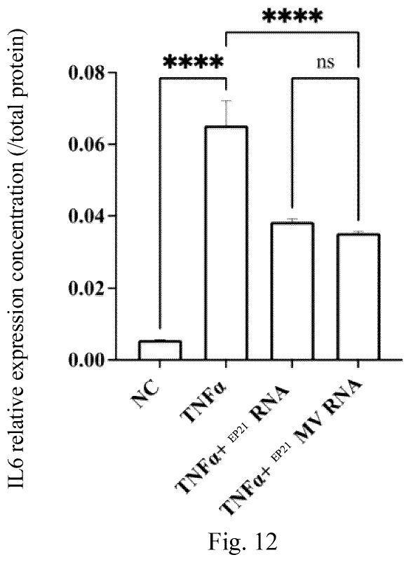

In order to test whether the present EP21 MVs can inhibit secretion of inflammatory cytokines for a long time, 2.5 ng/ml TNFα or 1.25 ng/ml IL1B is used to induce inflammation of retinal pigmented epithelial cells RPE-1 for 24 hours. A treatment group is added with 4.4×10⁶ particles of EP21 MVs at the same time to inhibit IL6 secretion induced by TNFα or IL1β. As shown in FIG. 11A, the present EP21 MVs can inhibit IL 6 secretion induced by TNFα. As shown in FIG. 11B, the present EP21 MVs can inhibit IL 6 secretion induced by IL1β.

In order to test whether RNA of the present EP21 MVs can inhibit secretion of inflammatory cytokines for a long time, introduce the strain EP21 RNA and EP21 MV RNA (1.5 μg respectively) into retinal pigmented epithelial cells RPE-1 (transfection) and use 2.5 ng/ml TNFα to induce inflammation for 24 hours. Then enzyme-linked immunosorbent assay (ELISA) is used to detect whether the cells after transfection can inhibit IL 6 secretion induced by TNFα. The ELISA contains the following steps. Dilute antibody (IL6) with 1×PBS in ELISA plate (4° C., overnight) and add blocking buffer to react for 1 hour. Add samples to conjugate for 2 hrs and add horseradish peroxidase (HRP) to react for a half hour. Then add 3,3′5,5′-Tetramethylbenzidine (TMB) substrate to generate colors. Wash the plate three times with 1×PBS between the two adjacent steps. According to color development of standards with different dilution concentrations, add IN HCL to terminate the reaction and read the absorbance at 450 nm. As shown in FIG. 12, the present strain EP21 RNA and EP21 MV RNA can also inhibit IL 6 secretion induced by TNFα.

### Embodiment 6: Effect of Eye Drops Containing EP21 MVs on Rat with TGF-β2 Induced Myopia

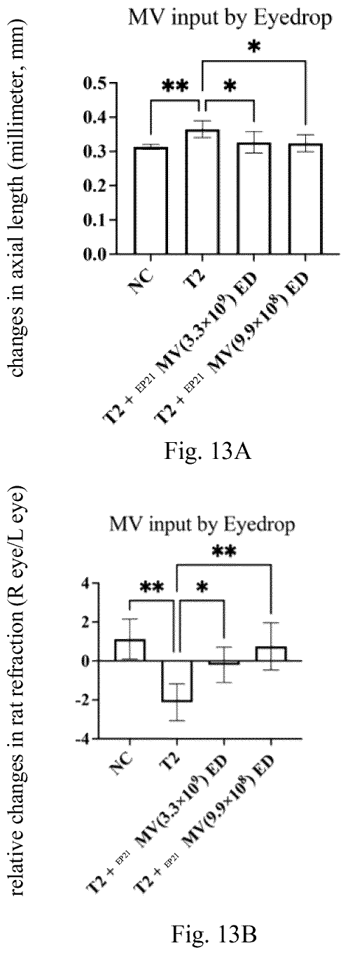

Use 3-week-old Brown Norway rat as test animals. After pre-test measurement of the animals, inject 250 ng/ml TGF-β2 to an upper eyelid of a right eye once a week to induce inflammation for 3 weeks. In a negative control group, inject balanced salt solution (BSS, eye wash buffer, used tor preparing injection medicines) and put in eye drops 1×BS for 3 weeks. In a positive control group, inject TGF-β2 and put in eye drops 1×PBS for 3 weeks. A first treatment group is injected with TGF-β2 and put in eye drops containing EP21 MVs for 3 weeks (applied with high concentration eye drops twice per week for 3 weeks with total amount of 3.3×10⁹ particles). A second treatment group is injected with TGF-β2 and put in eye drops containing EP21 MVs for 3 weeks (applied with low concentration eye drops seven times per week for 3 weeks with total amount of 9.9×10⁸ particles). Measure the axial length and the retraction before and after the treatment to verify changes among the respective groups. As shown in FIG. 13A and FIG. 13B, the results indicate that TGF-β2 significantly increases the axial length and changes the refraction of the rat. In the treatment groups, putting in the eye drops containing the EP21 MVs with different numbers of particles inhibits an excessive increase in the axial length and changes in the refraction.

### Embodiment 7: Effect of EP21 MVs on Rat with TGF-β2 Induced Myopia

### 1. Tail Vein Administration of EP21 MVs

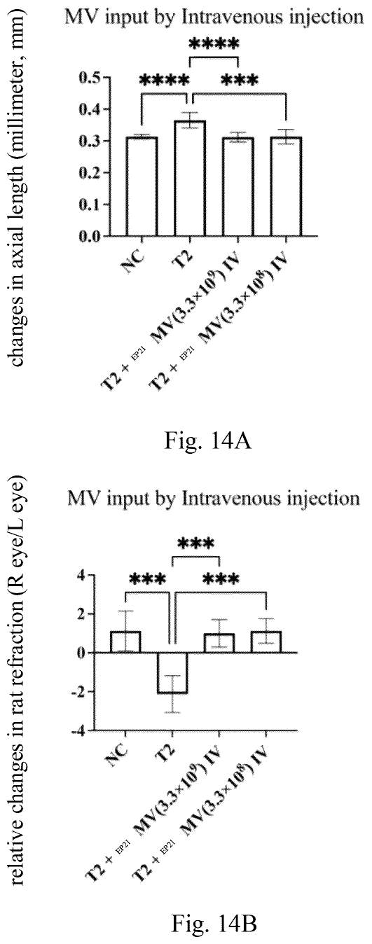

Use 3-week-old Brown Norway rat as test animals. After pre-test measurement of the animals, inject 250 ng/ml TGF-β2 to an upper eyelid of a right eye once a week to induce inflammation for 3 weeks. In a negative control group, inject balanced salt solution (BSS, eye wash buffer, used tor preparing injection medicines) and inject 1×PBS into the tail veins for 3 weeks. In a positive control group, inject TGF-β2 and inject 1×PBS into the tail veins for 3 weeks. A first treatment group is injected with TGF-β2 and applied with EP21 MVs by tail vein injection for 3 weeks (injected with high concentration twice per week for 3 weeks with total amount of 3.3×10⁹ particles). A second treatment group is injected with TGF-β2 and applied with EP21 MVs by tail vein injection for 3 weeks (injected with low concentration twice per week for 3 weeks with total amount of 3.3×10⁸ particles). Measure the axial length and the retraction before and after the treatment to verify changes among the respective groups. Refer to FIG. 14A and FIG. 14B, the results show that TGF-β2 significantly increases the axial length and changes the refraction of the rat. In the treatment groups applied with the EP21 MVs having different numbers of particles via the tail vein injections, an excessive increase in the axial length and changes in the refraction are both inhibited.

### 2. Intraperitoneal Administration of EP21 MVs

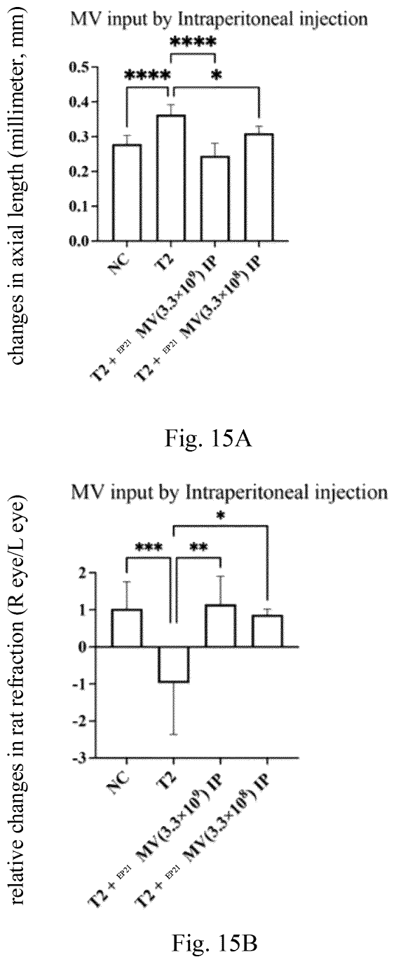

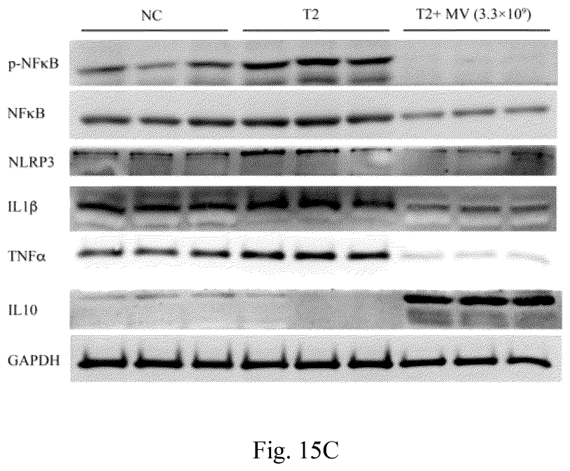

Use 3-week-old Brown Norway rat as test animals. After pre-test measurement of the animals, inject 250 ng/ml TGF-β2 to an upper eyelid of a right eye once a week to induce inflammation for 3 weeks. In a negative control group, inject balanced salt solution (BSS, eye wash buffer, used tor preparing injection medicines) and give an intraperitoneal injection of 1×PBS for 3 weeks. In a positive control group, inject TGF-β2 and give an intraperitoneal injection of 1×PBS for 3 weeks. A first treatment group is injected with TGF-β2 and given an intraperitoneal injection of EP21 MVs for 3 weeks (injected with high concentration twice per week for 3 weeks with total amount of 3.3×10⁹ particles). A second treatment group is injected with TGF-β2 and given an intraperitoneal injection of EP21 MVs for 3 weeks (injected with low concentration twice per week for 3 weeks with total amount of 3.3×10⁸ particles). Measure the axial length and the retraction before and after the treatment to verify changes among the respective groups. Take whole eyeballs as samples to perform western blotting and immunohistochemistry (IHC) analysis. As shown in FIG. 15A and FIG. 15B, the results show that TGF-β2 significantly increases the axial length and changes the refraction of the rat. In the treatment groups, administering the EP21 MVs with different numbers of particles by intraperitoneal injection inhibits an excessive increase in the axial length and changes in the refraction.

The samples of intraperitoneal administration of EP21 MVs of the embodiment 7 (the rat eyeballs) are added with RIPA lysis buffer (50 Mm Tris-HCl, 250 mMNaCl, 1% NP-40, 0.5% Sodium deoxycholate, 0.1% Sodium dodecyl sulfate, protease inhibitor and phosphatase inhibitor) and cut into pieces to perform western blotting. Refer to FIG. 15C, TGF-β2 activates expression of inflammatory targets including p-NFκB, NFκB, NLRP3, IL1B, and TNFα and reduces expression of anti-inflammatory target IL10. Thereby the intraperitoneal injection of the present EP21 MVs can inhibit activation of expression of inflammatory targets including p-NFκB, NFκB, NLRP3, IL1B, and TNFα and increase expression of anti-inflammatory target IL10.

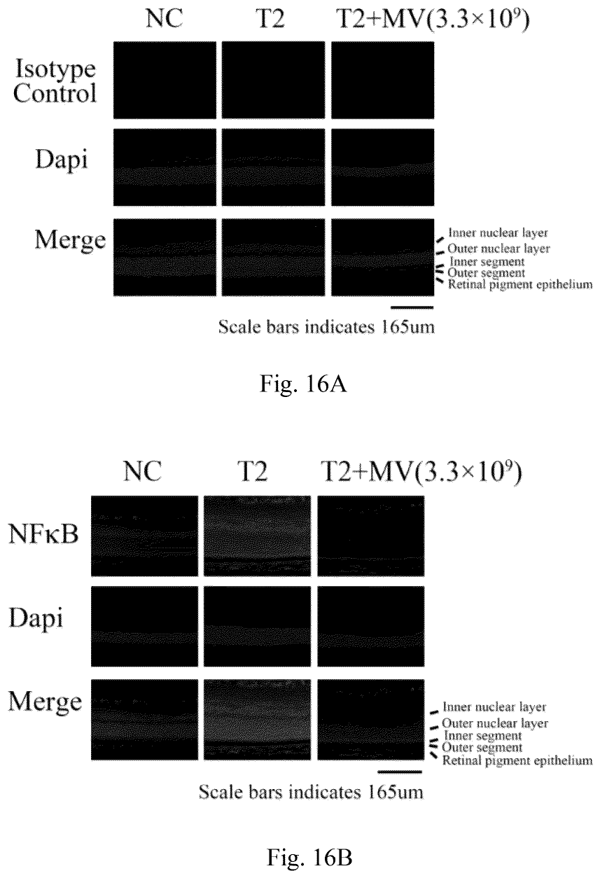

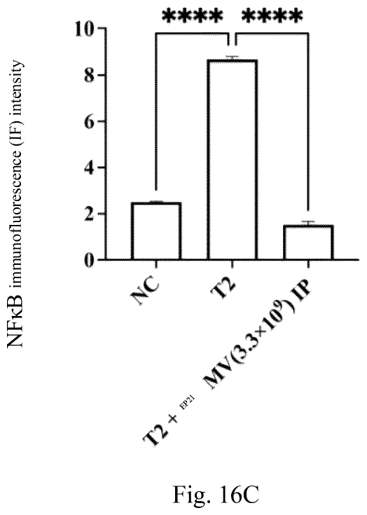

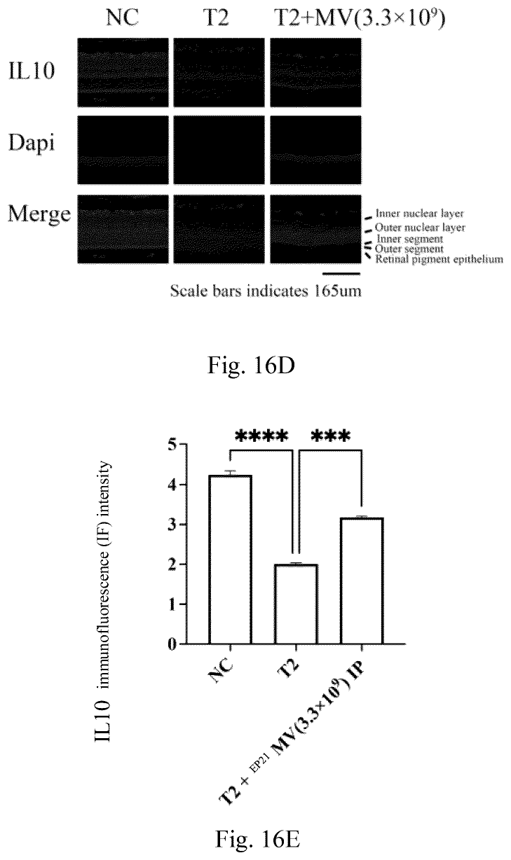

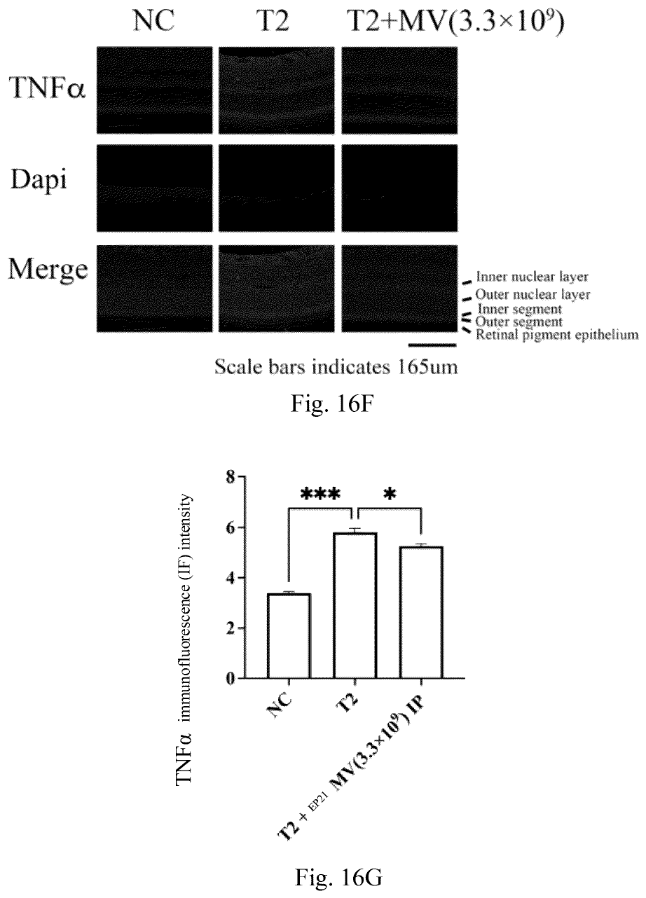

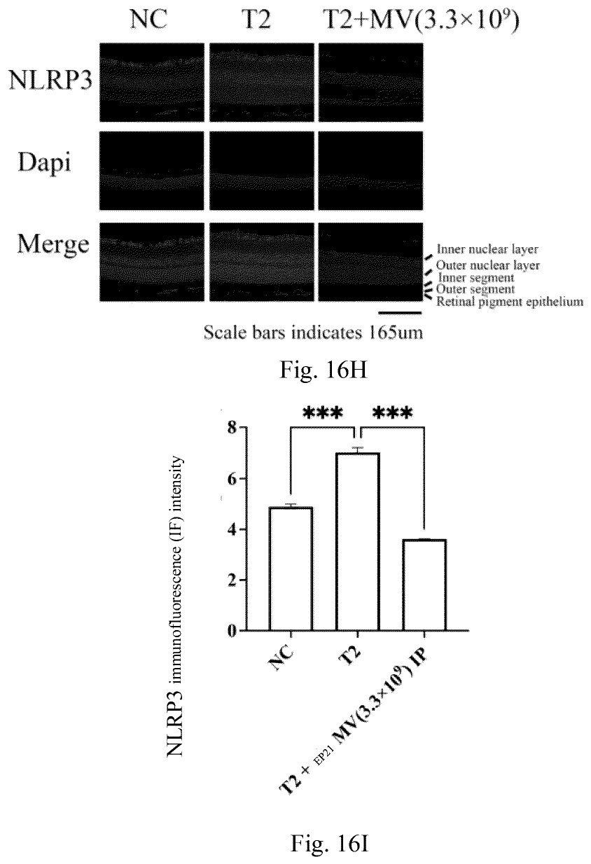

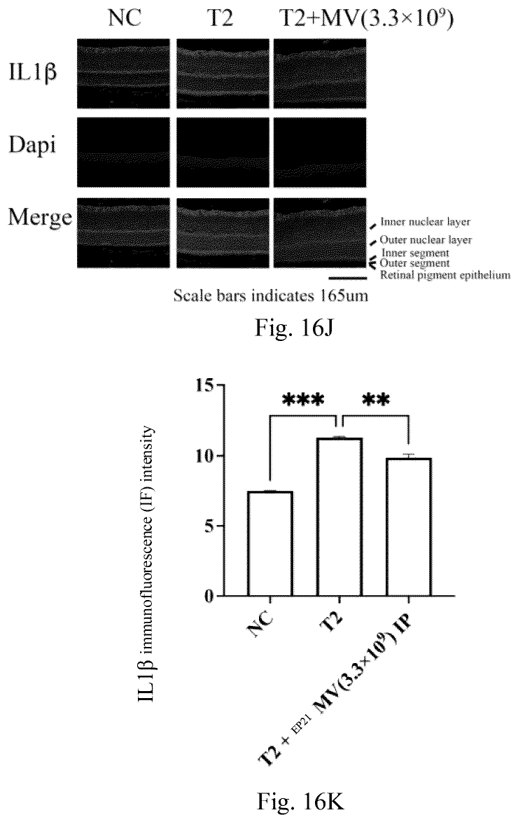

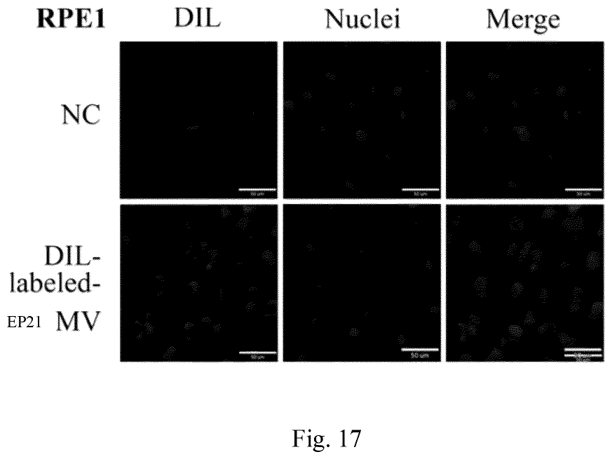

The samples of intraperitoneal administration of EP21 MVs of the embodiment 7 (the rat eyeballs) are analyzed by immunofluorescence and quantified using Image J. Then numerical values obtained are interpreted by one-way ANOVA analysis in GraphPad Prism. P value<0.05 is typically considered to be statistically significant. As the results shown in FIG. 16A-16K, TGF-β2 activates expression of inflammatory targets including p-NFκB, NFκB (FIG. 16B-16C), NLRP3 (FIG. 16H-16I), IL1ß (FIG. 16J-16K), and TNFα (FIG. 16F-16G) and reduces expression of anti-inflammatory target IL10 (FIG. 16D-16E). Thereby the intraperitoneal injection of the present EP21 MVs can inhibit activation of expression of inflammatory targets including p-NFκB, NFκB, NLRP3, IL1B, and TNFα and increase expression of anti-inflammatory target IL10.

### Embodiment 8: Phagocytosis Assay

Seed retinal pigmented epithelial cells RPE1 (1×10⁴) on a Chamber slide (PEZGS0816, MilliporeSigma™) and culture for about 16-18 hrs. After attachment of the cells, the EP21 MVs (4×10⁸) labelled with DIL and obtained in preparation embodiment 2 is used to treat the RPE1 cells for 24 hrs. Then use modular DMi8 inverted microscope (Leica/CCD, Andor ZYLA 4.2 Plus) for fluorescence excitation and observe EP21 MVs engulfed by cells. In vivo MV tracking uses Sprague-Dawley (SD) rats as test animals. Inject EP21 MVs (1×10¹⁰) labelled with DIL to the rat's tail. After 24 hours, kill the SD rat, collect eyeballs and fix tissues with formalin. Then dehydrate with 30% sucrose solution for 2 hrs, embed with optimal cutting temperature compound (OCT, FSC 22 Clear, 3801480, Leica), and freeze at −80° C. for storage. Use tissue chopper with a machine and a carrier both at −20° C. for preparation of slices from the tissue and a thickness of each slice is 12 μm. Use DAPI (1:1000) for nuclear staining, protected from light and react for 5 mins. After mounted with anti-bleaching reagent, use a fluorescence microscope for fluorescence excitation and taking images.

As shown in FIG. 17, the present EP21 MVs are engulfed by the retinal pigmented epithelial cell RPE1.

### Embodiment 9: Test of EP21 MVs to Pass Blood Retinal Barrier (BRB)

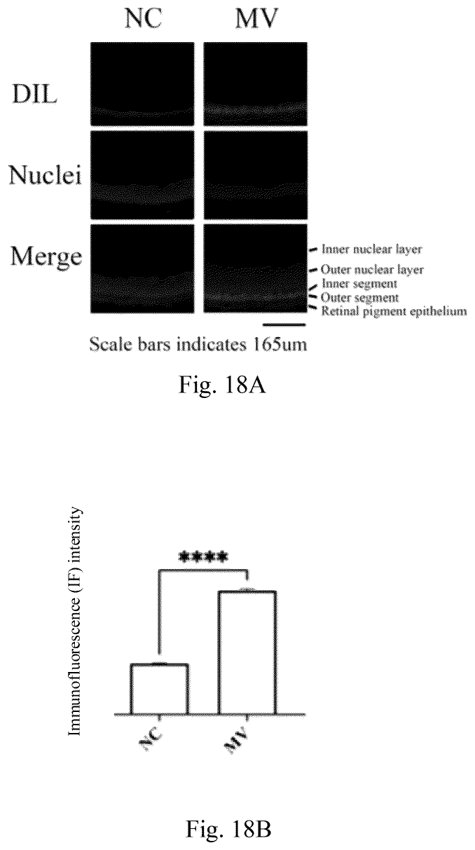

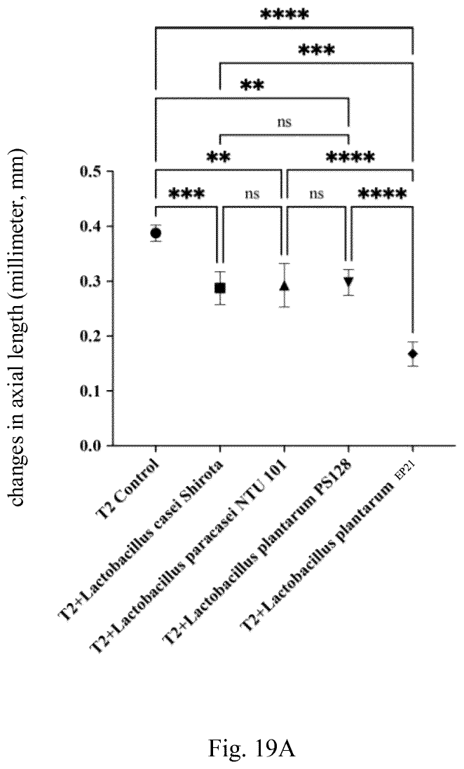

Inject EP21 MVs (1×10¹⁰) labelled with DIL to SD rat's tail. After 24 hours, kill the SD rat, collect eyeballs and fix tissues with formalin. Then dehydrate with 30% sucrose solution for 2 hrs, embed with optimal cutting temperature compound (OCT, FSC 22 Clear, 3801480, Leica), and freeze at −80° C. for storage. Use tissue chopper with a machine and a carrier both at −20° C. for preparation of slices from the tissue and a thickness of each slice is 12 μm. Use DAPI (1:1000) for nuclear staining, protected from light and react for 5 mins. After mounted with anti-bleaching reagent, use a fluorescence microscope for fluorescence excitation and taking images. As shown in FIG. 18A and FIG. 18B, the results show that the present EP21 MVs can pass BRB through blood flows.

### Embodiment 10: Test of Other Probiotics that Inhibit Inflammation

Use 3-week-old Brown Norway rat as test animals. After pre-test measurement of the animals, inject 250 ng/ml TGF-β2 to an upper eyelid of a right eye once a week to induce inflammation for 3 weeks (respectively Day 1, Day 8, and Day 15). In a negative control group, inject balanced salt solution (BSS, eye wash buffer, used tor preparing injection medicines) with tube-feeding of 1×PBS for 3 weeks. In a positive control group, inject TGF-β2 and 1×PBS for 3 weeks. A treatment group is injected with TGF-β2 and having tube feeding with probiotics on exponential phase (2.5-3×10⁹ CFU/0.1 ml/day) for 3 weeks. Besides the the EP21 in the predation embodiment 1, the rest three groups of probiotics are Lactobacillus casei Shirota, Lactobacillus paracasei NTU 101, and Lactiplantibacillus plantarum subsp. plantarum PS128. Measure the axial length and the retraction before and after the treatment to verify changes among the respective groups. After taking whole eyeballs as samples, perform western blotting and immunohistochemistry (IHC) analysis.

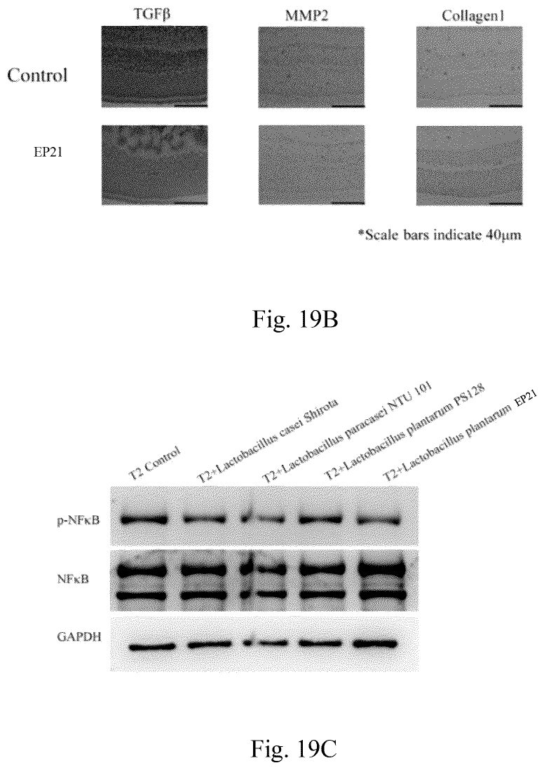

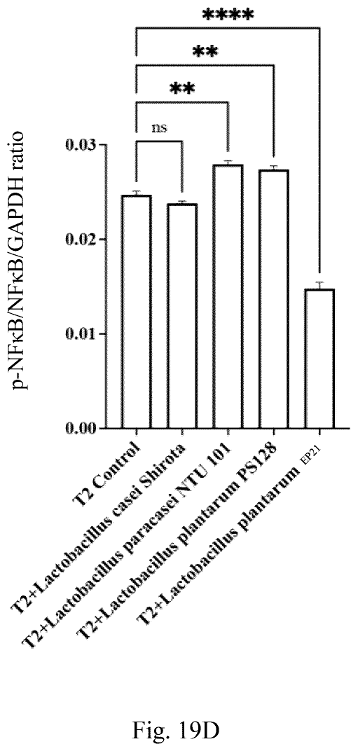

As the results shown in FIG. 19A, TGF-β2 significantly increases the axial length and changes the refraction of the rat. In the treatment group with the feeding of the EP21, an excessive increase in the axial length and changes in the refraction are inhibited. Moreover, during myopia progression, genes of the two tissue-remodeling proteins TGF-β and MMP2 are up-regulated. MMP2 is an extracellular matrix enzyme which degrades type I collagen (collagen 1) on the sclera. Along with development of myopia, the eyeball gradually elongates so that an image is formed in front of the retina when light comes into the eye. This causes myopia. FIG. 19B show results of immunohistochemistry (IHC) analysis used for detection of the amount of TGF-β, MMP2, and Collagen 1 of the retina. Color depth after color development represents the amount of the target protein on the retina. The control shown in FIG. 19B means the group treated with TGF-β2. The results show that expression of TGF-β2 and MMP2 is increased while expression of Collagen 1 is decreased in TGF-β2-induced group with myopia. After being fed with the EP21, expression of TGF-β2 and MMP2 is decreased while expression of Collagen 1 is increased. According to the above changes, it is learned that the feeding of the EP21 improves TGF-β2-induced myopia. The results of western blotting are shown in FIG. 19C and FIG. 19D. The feeding of the EP21 according to the present invention significantly inhibits activation of the transcription factor NFκB. The results of the above tests show that the EP21 strain of the present invention is anti-inflammatory and able to slow down myopia progression compared with other lactic acid bacteria (lactobacillus).

1. A Lactiplantibacillus plantarum subsp. plantarum strain EP21 with an accession number of BCRC (Biosource Collection and Research Center (BCRC)) 911210 for use in improvement of eye diseases, wherein the strain includes a segment of sequence shown in Seq ID No. 3.

2. A use of the Lactiplantibacillus plantarum subsp. plantarum strain EP21 as claimed in claim 1 for preparing a pharmaceutical composition used to improve eye diseases.

3. The use as claimed in claim 2, wherein the pharmaceutical composition further includes a pharmaceutically acceptable excipient, carrier, adjuvant and food additive.

4. The use as claimed in claim 2, wherein a dosage form of the pharmaceutical composition includes solution, suspension, capsule, pill, lozenge, troche, powder, freeze-dried powder, lotions, emulsions, suppository, slurry, and ointment.

5. The use as claimed in claim 2, wherein the pharmaceutical composition is applied to recipients by oral administration.

6. The use as claimed in claim 5, wherein an effective dose of the pharmaceutical composition is application of 1×10⁶ cfu/kg to 1×10¹⁰ cfu/kg to recipients per day.

7. The use as claimed in claim 2, wherein the eye diseases include myopia, dry eye syndrome (DES), eye fatigue, keratitis, macular degeneration, retinitis pigmentosa (RP), proliferative diabetic retinopathy (PDR), ischemic retinopathy, choroidal neovascularization, glaucoma, systemic lupus erythematosus (SLE), and Sjögren's syndrome (SS).

8. The use as claimed in claim 7, wherein the ischemic retinopathy includes diabetic retinopathy (DR), retinopathy of prematurity (ROP), central retinal vein occlusion (CRVO), and ischemic optic neuropathy (ION).

9. A composition containing bacterial membrane vesicles used for improvement of eye diseases comprising bacterial membrane vesicles (MVs) derived from the Lactiplantibacillus plantarum subsp. plantarum strain EP21 as claimed in claim 1.

10. The composition as claimed in claim 11, wherein a vesicle diameter of the MV is ranging from 100 nm to 300 nm.

11. A use of the compositions containing bacterial membrane vesicles as claimed in claim 9 for preparing a pharmaceutical composition used to improve eye diseases.

12. The use as claimed in claim 11, wherein the pharmaceutical composition further includes a pharmaceutically acceptable excipient, carrier, adjuvant and food additive.

13. The use as claimed in claim 11, wherein a dosage form of the pharmaceutical composition includes solution, suspension, capsule, pill, lozenge, troche, powder, freeze-dried powder, lotions, emulsions, suppository, slurry, and ointment.

14. The use as claimed in claim 11, wherein the pharmaceutical composition is applied to recipients by injections or putting in eye drops.

15. The use as claimed in claim 14, wherein the injections include intravenous injections (IV), intraperitoneal injections (IP), and tail vein injections.

16. The use as claimed in claim 14, wherein an effective dose of the pharmaceutical composition is application of 1×10⁵ particles/kg to 1×10¹¹ particles/kg to recipients per day.

17. The use as claimed in claim 11, wherein the eye diseases include myopia, dry eye syndrome (DES), eye fatigue, keratitis, macular degeneration, retinitis pigmentosa (RP), proliferative diabetic retinopathy (PDR), ischemic retinopathy, choroidal neovascularization, glaucoma, systemic lupus erythematosus (SLE), and Sjögren's syndrome (SS).

18. The use as claimed in claim 17, wherein the ischemic retinopathy includes diabetic retinopathy (DR), retinopathy of prematurity (ROP), central retinal vein occlusion (CRVO), and ischemic optic neuropathy (ION).

19. A use of the compositions containing bacterial membrane vesicles as claimed in claim 10 for preparing a pharmaceutical composition used to improve eye diseases.

20. The use as claimed in claim 19, wherein the pharmaceutical composition further includes a pharmaceutically acceptable excipient, carrier, adjuvant and food additive.

21. The use as claimed in claim 19, wherein a dosage form of the pharmaceutical composition includes solution, suspension, capsule, pill, lozenge, troche, powder, freeze-dried powder, lotions, emulsions, suppository, slurry, and ointment.

22. The use as claimed in claim 19, wherein the pharmaceutical composition is applied to recipients by injections or putting in eye drops.

23. The use as claimed in claim 22, wherein the injections include intravenous injections (IV), intraperitoneal injections (IP), and tail vein injections.

24. The use as claimed in claim 22, wherein an effective dose of the pharmaceutical composition is application of 1×10⁵ particles/kg to 1×10¹¹ particles/kg to recipients per day.

25. The use as claimed in claim 19, wherein the eye diseases include myopia, dry eye syndrome (DES), eye fatigue, keratitis, macular degeneration, retinitis pigmentosa (RP), proliferative diabetic retinopathy (PDR), ischemic retinopathy, choroidal neovascularization, glaucoma, systemic lupus erythematosus (SLE), and Sjögren's syndrome (SS).

26. The use as claimed in claim 25, wherein the ischemic retinopathy includes diabetic retinopathy (DR), retinopathy of prematurity (ROP), central retinal vein occlusion (CRVO), and ischemic optic neuropathy (ION).


SEQUENCE LISTING (3 sequences)

SEQ ID NO: 1 - DNA, 20 bp, unidentified
  source 1..20: mol_type = other DNA; note = artificial sequence
agagtttgatcctggctcag

SEQ ID NO: 2 - DNA, 20 bp, unidentified
  source 1..20: mol_type = other DNA; note = artificial sequence
cggttaccttgttacgactt

SEQ ID NO: 3 - DNA, 1050 bp, Lactobacillus plantarum
  source 1..1050: mol_type = other DNA
ggcgggggcgggtgactatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattctaagctacgcggagaacccttaccaaggtcttgacatactatgcaaatctaagagattagacgtttccctttcggggacatgggatacaggtggtg